(19) United States
(12) Patent Application Publication
(10) Pub. No.: US 2016/0363600 A1
(43) Pub. Date: Dec. 15, 2016

(54) FLUIDICS DEVICES FOR INDIVIDUALIZED COAGULATION MEASUREMENTS AND ASSOCIATED SYSTEMS AND METHODS

(71) Applicant: University of Washington, Seattle, WA (US)

(72) Inventors: Nathan J. SNIADECKI, Seattle, WA (US); Nathan J. WHITE, Seattle, WA (US); Ari KARCHIN, Seattle, WA (US); Lucas H. TING, Seattle, WA (US)

(21) Appl. No.: 14/902,547

(22) PCT Filed: Jun. 26, 2014
(86) PCT No.: PCT/US2014/044448

§ 371 Date: Dec. 31, 2015

Related U.S. Application Data
(60) Provisional application No. 61/839,723, filed on Jun. 26, 2013.

(51) Int. Cl. G01N 33/86; B01L 3/00

(52) U.S. Cl. CPC G01N 33/86; B01L 3/502715; B01L 3/502738; B01L 2300/123; B01L 2300/0627; B01L 2300/0819; B01L 2300/0832; G01N 2800/224; B01L 2300/025

(57) ABSTRACT

The present technology relates generally to fluidics devices for measuring platelet coagulation and associated systems and methods. In some embodiments, a fluidics device includes an array of microstructures including pairs of generally rigid blocks and generally flexible posts. The fluidics device further includes at least one fluid channel configured to accept the array. The fluidics device can further include a measuring element configured to measure a degree of deflection of one or more of the flexible posts in the array. In some embodiments, the fluidics device comprises a handheld device and usable for point of care testing of platelet forces and coagulation.

### CROSS-REFERENCE TO RELATED APPLICATIONS

This application claims the benefit of U.S. Provisional Application No. 61/839,723, filed Jun. 26, 2013, titled “Device and Method for Multiplexed Patient Specific Platelet Thrombosis and Fibrinolysis Testing with Internal Controls,” which is incorporated herein by reference in its entirety.

### TECHNICAL FIELD

The present technology relates generally to fluidics devices for making individualized coagulation measurements, and associated systems and methods.

### BACKGROUND AND SUMMARY

Trauma accounts for one in ten, or approximately five million, deaths annually worldwide and consumes over $135 billion in U.S. annual healthcare expenditure. The majority of trauma deaths occur within the first hour after injury (the “golden hour”) from uncontrolled hemorrhaging. Trauma-induced coagulopathy (TIC), or impaired clot formation, contributes to this uncontrolled hemorrhaging and is present in about 25% of trauma patients. Uncontrolled hemorrhaging during TIC may not be readily apparent to the response team, as often times the hemorrhaging occurs internally. TIC occurs almost immediately after injury and is associated with a several fold increased incidence of multi-organ failure, intensive care utilization, and death. This makes early diagnosis and treatment of TIC a top priority in emergency medicine.

Under normal conditions, a multi-factorial process drives the formation of clots during hemorrhage to achieve hemostasis (cessation of bleeding). As shown schematically in FIG. 1, clots are dynamic structures comprised mainly of platelets P and a mesh of fibrin fibers F. In a first stage of hemostasis, the platelets P adhere to a wound site and to one another, and contract (individually or in the aggregate) to form a platelet plug. As such, the formation of a clot structure is mediated, at least in part, by platelet P contractile forces. In a second stage of hemostasis, the activated platelets P generate the protease thrombin (not shown) that converts soluble fibrinogen into fibrin fibers F at the wound site. The fibrin fibers F form around the plug to hold the platelets P together and prevent dislodgement of the newly formed clot.

At least three clot parameters—clot strength, clot onset, and clot lysis—are recognized as important for achieving and maintaining hemostasis. As used herein, “clot strength” refers to the peak clot contractile force, “clot onset” refers to the time it takes for a clot to form, and “clot lysis” refers to the decrease in clot strength after peak contraction. TIC impacts one or more of these clot parameters which ultimately impairs stable clot formation. For example, TIC can reduce clot strength, as TIC often leads to hypoperfusion (i.e., insufficient blood supply to vital organs), and hypoperfusion leads to reduced thrombin generation and thus reduced fibrin F formation around the platelet plug. TIC can also enhance or accelerate clot lysis by increasing the availability of tissue plasminogen activator (tPA), a protein that converts plasminogen to plasmin (i.e., the enzyme responsible for clot breakdown by breaking down the fibrin F mesh). Hypoperfusion also accelerates clot lysis due to the resulting build-up of lactic acid and reduction in pH levels.

Measuring clot formation to detect TIC is currently accomplished by the use of thrombelastography (TEG) devices that measure viscoelasticity to assess clot formation and report clot parameters, such as clot strength, clot onset, and clot lysis. Although the measurements taken from TEG devices have been shown to be more sensitive and accurate indicators of clotting than those taken using other conventional tests (e.g., prothrombin time (PT), activated partial thromboplastin time (aPTT), international normalized ratio (INR), etc.), TEG devices are large (generally used as bench-top devices), expensive, and sensitive to movement. Accordingly, TEG devices are not appropriate as true point-of-care devices capable of determining a clot parameter value and/or making a measurement at the patient's bedside where early detection of TIC is needed. Moreover, TEG devices require 20-30 minutes to produce a reading, which means that a first reading from either device is typically not available to the treatment clinician(s) until well past the golden hour. Given that approximately one third of patients arriving to the ER die within 15 minutes of arrival, waiting 20-30 minutes for a reading from a TEG device is unsatisfactory for diagnosing TIC.

The current treatment for patients diagnosed with TIC is a transfusion of blood components, such as plasma, platelets, red blood cells (RBCs), and others. Plasma is transfused to increase the concentration of clotting proteins and fibrinogen (the precursor for fibrin), platelets are transfused to increase the number of healthy platelets available, and RBCs are transfused to replace blood loss due to severe hemorrhage and also to restore oxygen delivery to organs and tissues. Currently, the generally accepted “best practice” consists of a 1:1:1 ratio of plasma, platelets, and RBCs, regardless of the relative value of the patient's clot parameters. Such potentially inaccurate or uninformed diagnoses of TIC is concerning, as there are high risks associated with transfusion of blood components, including multiple organ failure, acute respiratory distress syndrome (ARDS), increased infection, and increased mortality.

Accordingly, there exists a need for improved devices and methods for measuring coagulation of a patient.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 is a schematic representation of the stages of clot formation within a blood vessel.

FIG. 2A shows a clot analyzing system configured in accordance with an embodiment of the present technology.

FIG. 2B is an enlarged view of a portion of a fluidics device of the clot analyzing system in FIG. 2A showing an array of sensing units configured in accordance with an embodiment of the present technology.

FIG. 2C is an enlarged view of a sensing unit of the array shown in FIG. 2B.

FIG. 3 is a schematic side view of a chamber of the fluidics device shown in FIG. 2A configured in accordance with an embodiment of the present technology.

FIGS. 4A-4D are time-lapsed top views of a sensing unit during delivery of a biological sample in accordance with an embodiment of the present technology.

FIG. 5 is a top view of an individual sensing unit showing aggregated platelets contracting to bend the micropost towards the microblock in accordance with an embodiment of the present technology.

FIG. 6 is a graph showing clotting forces versus time.

FIG. 7 is a schematic side view of a measuring element comprising an optical component and configured in accordance with an embodiment of the present technology.

FIG. 8A is a side view of a plurality of microposts and a measuring element comprising a magnetic component configured in accordance with an embodiment of the present technology. In FIG. 8A, the plurality of microposts are shown before deflection and configured in accordance with an embodiment of the present technology.

FIG. 8B is a side view of the measuring element and microposts in FIG. 8A. In FIG. 8B, the microposts are shown in a deflected state and configured in accordance with an embodiment of the present technology.

FIG. 9 is a graph showing spin-valve voltage versus displacement for a deflected micropost configured in accordance with an embodiment of the present technology.

FIG. 10 is a top view of a fluidics device having multiple arrays and configured in accordance with the present technology.

### DETAILED DESCRIPTION

The present technology describes various embodiments of devices, systems, and methods for measuring one or more clot parameters. In one embodiment, for example, the system includes a plurality of arrays of microstructures, wherein each microstructure includes a generally rigid structure and a generally flexible structure. A first array can be configured to be in fluid connection with a first clotting agent, a second array can be configured to be in fluid connection with a second clotting agent different than the first clotting agent, and a third array is not in fluid connection with the first clotting agent or the second clotting agent. The system can further include a plurality of fluid channels configured to receive a biological sample flowing therethrough. At least a portion of the fluid channels can be individually sized to accept one of the arrays. In some embodiments, the system can include a measuring element that is configured to detect a degree of deflection of one or more of the flexible structures in one or more of the arrays.

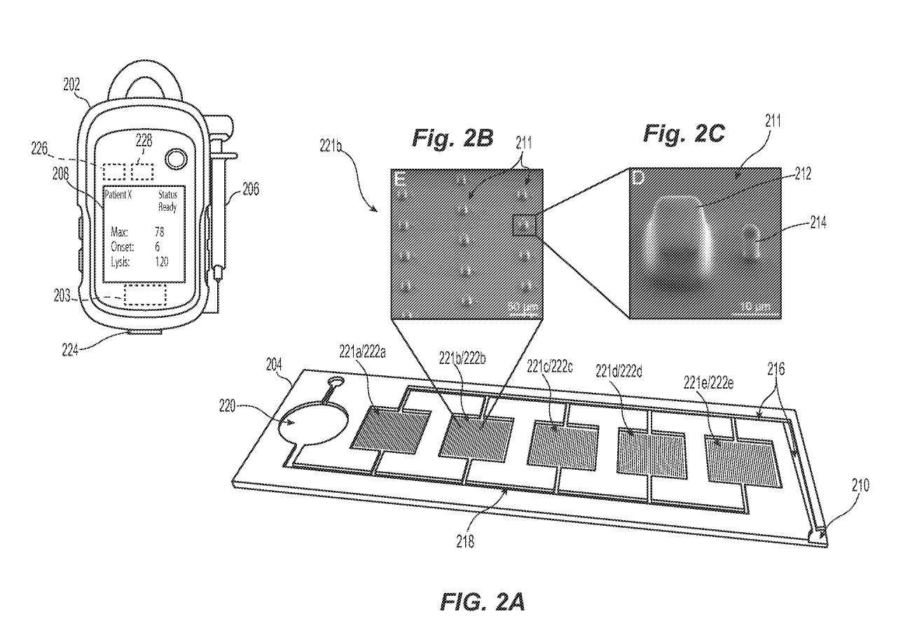

Specific details of several embodiments of the technology are described below with reference to FIGS. 2A-10. Other details describing well-known structures and systems often associated with TEG devices, biomedical diagnostics, immunoassays, etc. have not been set forth in the following disclosure to avoid unnecessarily obscuring the description of the various embodiments of the technology. Many of the details, dimensions, angles, and other features shown in FIGS. 2A-10 are merely illustrative of particular embodiments of the technology. Accordingly, other embodiments can have other details, dimensions, angles, and features without departing from the spirit or scope of the present technology. A person of ordinary skill in the art, therefore, will accordingly understand that the technology may have other embodiments with additional elements, or the technology may have other embodiments without several of the features shown and described below with reference to FIGS. 2A-10.

### I. SELECTED EMBODIMENTS OF CLOT ANALYZING DEVICES, SYSTEMS AND METHODS FOR MEASURING MICROPOST DEFLECTION

FIG. 2A shows one embodiment of a clot analyzing system 200 configured in accordance with the present technology. As shown in FIG. 2A, the clot analyzing system 200 can include a fluidics device 204, an analyzer 202, and an introducer 206. The introducer 206 can be a pressurized conduit (e.g., a syringe, a syringe pump, etc.) that is configured to collect and/or hold a biological sample (e.g., blood) and deliver the biological sample to the fluidics device 204. The biological sample can include whole blood, platelets, endothelial cells, circulating tumor cells, cancer cells, fibroblasts, smooth muscle cells, cardiomyocytes, red blood cells, white blood cells, bacteria, megakaryocytes, and/or fragments thereof. The introducer 206 can be detachably coupled to the analyzer 202 (as shown in FIG. 2A), or in some embodiments the introducer 206 can be a standalone device. Before, during, and/or after delivery of the biological sample to the fluidics device 204, the fluidics device 204 can be coupled to the analyzer 202 (e.g., via a port 224). The analyzer 202 can be a handheld device configured to measure one or more clot parameters present in one or more clots formed by the biological sample on the fluidics device 204. As described in greater detail below, the analyzer 202 can then provide an individualized measurement of one or more clot parameters and, based on the individualized measurement, determine a specialized diagnosis and/or treatment.

The fluidics device 204 can be a disposable microfluidic card having a network of microchannels and chambers configured to receive a biological sample (e.g., blood) flowing therethrough. In the embodiment illustrated in FIG. 2A, the fluidics device 204 includes an inlet port 210, an inlet channel 216, an outlet channel 218, a plurality of chambers (identified individually as first through fifth chambers 222a-e; referred to collectively as chambers 222), and an outlet reservoir 220. The inlet port 210 can be fluidly coupled to the inlet channel 216, and separate branches of the inlet channel 216 can be fluidly coupled to each of the chambers 222. The chambers 222 can be arranged in parallel such that the biological sample divides into as many portions as there are chambers 222, and each portion only flows through a single chamber before being routed to the outlet reservoir 220 via the branches of the outlet channel 218. Moreover, because of this arrangement, the biological sample flows through each of the chambers 222 almost simultaneously or near simultaneously. Simultaneous or near simultaneous flow through the plurality of chambers 222 can be advantageous for later comparison of clot parameters between the chambers 222, such as clot onset.

It will be appreciated that although the fluidics device 204 is shown having five chambers 222a-e, in other embodiments the fluidics device 204 can have more or fewer than five chambers (e.g., two, three, four, six, seven, etc.). Likewise, the fluidics device 204 can have any number of ports and/or channels, and the ports, channels, and chambers can be arranged in a variety of configurations. Additionally, although the fluidics device 204 is generally disposable, the fluidics device 204 can receive multiple discrete biological samples (from the same patient) and/or can be analyzed by the analyzer 202 more than once.

FIG. 2B is an enlarged view of a portion of the second chamber 222b of FIG. 2A, and FIG. 2C is an enlarged view of a portion of FIG. 2B. Referring to FIGS. 2A-2C together, each chamber 222 can include an array (identified individually as first through fifth arrays 221a-e; referred to collectively as arrays 221) of sensing units 211. The sensing units 211 can be arranged within the respective array 221a-e such that individual sensing units 211 in adjacent rows are offset from one another (as shown in FIG. 2B). In other words, the sensing units 211 can be arranged such that no sensing unit 211 is directly aligned with another sensing unit 211 in the immediately adjacent row. This configuration is expected to reduce the downstream effects of flow disturbances caused by upstream sensing units 211.

As best shown in FIG. 2C, each sensing unit 211 can include a generally rigid structure, such as a microblock 212 and a generally flexible structure, such as a micropost 214. The micropost 214 can be positioned downstream of the microblock 212 and in general alignment with a center line of the microblock 212. In certain embodiments, the micropost 214 can be positioned within about 8 μm (measured from edge to edge) of the microblock 212 so that biological sample components (e.g., cells) that aggregate on the microblock 212 are able to bridge the gap between the microblock 212 and the micropost 214. In other embodiments, the micropost 214 and the microblock 212 may be spaced apart by a greater or smaller distance depending upon the size of the biological components being analyzed.

The microblocks 212 can have a generally rectangular shape, and in some embodiments (including FIG. 2C), the microblocks 212 can have rounded edges and corners. In other embodiments, the microblocks 212 can have any suitable shape, size and/or configuration (e.g., a circular shape, a polyhedral shape, a sphere, etc.). In some embodiments, the individual microblocks 212 can have a length between about 10 μm and about 30 μm (e.g., about 20 μm), a width between about 5 μm and about 15 μm (e.g., about 10 μm), and a height between about 10 μm and about 20 μm (e.g., about 15 μm). The microposts 214 can have a generally cylindrical shape. In other embodiments, the microposts 214 can have any suitable shape, size and/or configuration (e.g., a circular shape, a polyhedral shape, a sphere, etc.). In some embodiments, the individual microposts 214 can have a diameter between about 2 μm and about 6 μm (e.g., about 4 μm), and a height between about 10 μm and about 20 μm (e.g., about 15 μm). The pairs of microblocks 212 and microposts 214 can have the same or different dimensions (e.g., heights) within the individual arrays 221 or chambers 222.

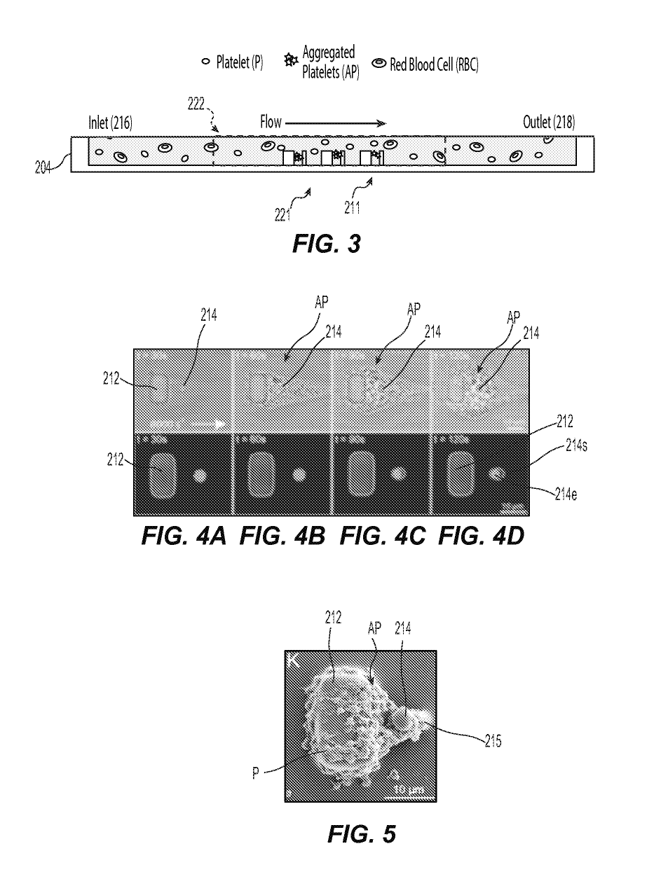

FIG. 3 is a schematic side view of one of the chambers 222 of the fluidics device 204 of FIG. 2A showing a biological sample, such as blood, flowing over one of the sensing unit arrays 221. FIGS. 4A-4D are time lapsed top views of one of the sensing units 211 shown in FIG. 3. The introducer 206 (FIG. 2A) can be configured to deliver the biological sample to the fluidics device 204 such that the biological sample flows over and around the individual sensing units 211 of the arrays 221. In some embodiments, the introducer 206 can be configured to deliver the biological sample at a flow rate sufficient to generate a shear rate at or near the sensing units 211 between about 2000 s-1 and about 12000 s-1 (e.g., 2000 s-1, 5000 s-1, 8000 s-1, 12000 s-1, etc.). In a particular embodiment, the introducer 206 is configured to maintain the desired flow rate for the duration of delivery (e.g., about 40 seconds to about 120 seconds).

Referring to FIGS. 3 and 4A-4B together, as the biological sample flows over the sensing units 211, each microblock 212 acts as a flow obstruction and causes an eddy. The eddy produces a high shear rate at the outermost top edges of the microblock 212 which activates the platelets P within the passing blood sample. The activated platelets P then bind to the microblock 212 (and to one another) as the platelets begin to aggregate. As shown in FIGS. 4B-4D, as an aggregation AP of platelets P grows larger in size, some of the platelets P breach the interstitial space between the microblock 212 and the micropost 214. For example, dual strands of collecting platelets P tend to form at the downstream corners of the microblock 212. As the platelet strands accumulate in length, the passing fluid pushes the strands inwardly and into contact with the micropost 214, thereby forming a mechanical bridge between the microblock 212 and the micropost 214. As more biological sample flows through the chamber 222, more platelets P accumulate and fill in the space between the microblock 212 and the micropost 214. In some embodiments, the microblock 212 and/or micropost 214 can be at least partially coated with at least one binding element (e.g., proteins, glycans, polyglycans, glycoproteins, collagen, etc) to improve and/or facilitate attachment of the platelets P to the microblock 212 and/or micropost 214.

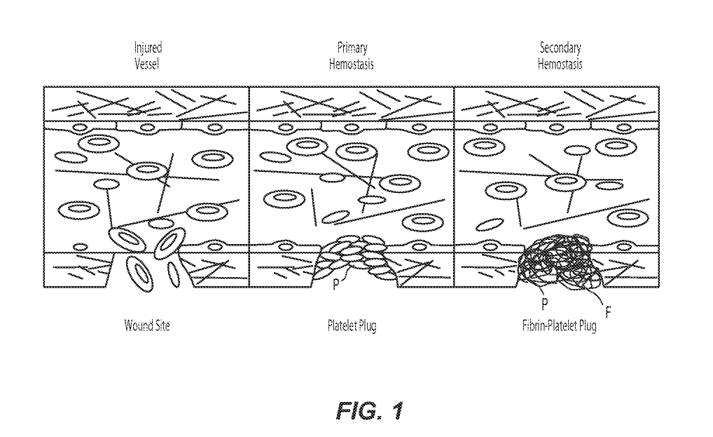

As discussed with reference to FIG. 1, during hemostasis the platelets P contract, both individually and en masse. Unlike the flexible micropost 214, the rigid microblock 212 does not bend despite its greater surface area and greater drag profile. Thus, when the platelets P contract, the platelets P bend the micropost 214 towards the microblock 212. For example, the confocal image (bottom image) of FIG. 4D shows that after 120 seconds of biological sample flow, the tip or top portion of the micropost (labeled 214e) is nearer (e.g., about 4 μm) to the microblock 212 than the top portion of the micropost when the flow began (labeled 214s). Likewise, the scanning electron microscope (SEM) micrograph of FIG. 5 shows the tip of the micropost 214 is bent away from a base portion 215 of the micropost 214.

Devices, systems and methods of the present technology for measuring and/or determining micropost deflection and determining a clot parameter value are described below.

a. Selected Embodiments of Devices, Systems and Methods for Determining Micropost Deflection

Referring back to FIG. 2A, the system 200 can further include a measuring element 203 for measuring and recording micropost deflection. The measuring element 203 can be carried by and/or contained within the analyzer 202 such that when the fluidics device 204 is at least partially inserted into the analyzer 202 (e.g., via the port 224), the measuring element 203 is positioned adjacent the fluidics device 204 to facilitate micropost deflection detection and/or deflection measurements. In other embodiments (not shown), the measuring element 203 is carried by the analyzer 202, but spaced apart from the fluidics device 204 and/or port 224. In yet other embodiments, the measuring element 203 can be a standalone device that can be physically or wirelessly coupled to the analyzer 202.

The measuring element 203 can be coupled to the analyzer 202 and, based on the measured micropost deflection, the analyzer 202 can determine a value for one or more clot parameters. The analyzer 202 can include a processor 226 and memory 228 having program instructions that, when executed by processor 226, cause the analyzer 202 to measure and record deflection data and analyze the measured data to determine the value of one or more clot parameters. The memory 228 may include any volatile, non-volatile, fixed, removable, magnetic, optical, or electrical media, such as a RAM, ROM, CD-ROM, hard disk, removable magnetic disk, memory cards or sticks, NVRAM, EEPROM, flash memory, and the like. The analyzer 202 can also indicate the current, measured value for one or more clot parameters to a clinician via a display 208 (FIG. 2A).

In a particular embodiment, the measuring element 203 can include an optical detection component that is configured to optically measure micropost deflection, such as a phase contrast microscope, a fluorescence microscope, a confocal microscope, or a photodiode. For example, FIG. 7 is a schematic side view of one embodiment of an optical measuring element 205 configured in accordance with the present technology. The fluidics device 204 can be positioned between a first portion 205a and a second portion 205b of the optical measuring element 205. In a particular embodiment, the fluidics device 204 can be inserted into a slot 296 in the optical measuring element 205 (and/or the analyzer 202 (e.g., via the port 224 (FIG. 2A)). The first portion 205a can be adjacent a first side of the slot 296, and the second portion 205b can be adjacent a second side of the slot 296 opposite the first side. The surfaces of the first and/or second side of the slot 296 can include first and second windows 298, 292, respectively, that are transparent or generally transparent. In other embodiments, the fluidics device 204 and/or the slot 296 can be positioned adjacent the first portion 205a and the second portion 205b without being between the first portion 205a and the second portion 205b. However, it is believed that a linear arrangement of the first portion 205a, the fluidics device 205b, and the second portion 205a can be advantageous as such an arrangement requires less space within the analyzer 202 (FIG. 2A).

Referring still to FIG. 7, the first portion 205a of the optical measuring element 205 can include a light source 280, an excitation filter 282, and a first focuser 284 comprised of a plurality of lenses (identified individually as first through third lenses 284a-284c). In other embodiments, the first focuser 284 can include more or fewer than three lenses (e.g., one, two, four, five, etc.). The light source 280 can be a mercury-lamps or xenon arc or another suitable light source used in fluorescence microscopy, such as lasers and LEDs. The second portion 205b of the optical measuring element 205 can include a second focuser 286 (labeled individually as first and second lenses 286a, 286b), an emission filter 288, and an optical detector 290. In other embodiments, the second focuser 286 can include more or fewer than two lenses (e.g., one, three, four, five, etc.). The optical detector 290 can be a camera, a photodiode, or any other suitable optical detection device.

In operation, the fluidics device 204 can be positioned at least partially within the slot 296, as shown in FIG. 7. The fluidics device 204 can be positioned directly on the window 292, or in other embodiments the fluidics device 204 can be carried by a transparent or generally transparent carrier 294 that can be positioned directly on the window 292, as shown in FIG. 7. The light source 280 can be manually or automatically triggered (via a sensor in the slot 296 coupled to the processor 226) to emit radiation toward the fluidics device 204. Only a particular wavelength of the emitted radiation passes through the excitation filter 282 and is focused on the array(s) 221 of sensing units 211 by the first focuser 284 (before delivering the biological sample to the device 204, the microblocks 212 and/or microposts 214 can be labeled with a fluorescent substance that specifically reacts to the particular, passed wavelength). As the particular wavelength collides with the atoms of the fluorescent substance on the micropost 214 and/or microblock 212, the atoms are excited to a higher energy level. When these atoms relax to a lower energy level, they emit light. The fluidics device 204 can be made of a transparent or generally transparent material (such as polydimethylsiloxane (PDMS)) such that the emitted light passes through fluidics device 204 (and carrier 294), through the window 292, and into the second portion 205b.

The emitted light is then focused by the second focuser 286. To become visible, the emission filter 288 separates the emitted light from the other much brighter radiation and thus only passes a lower, visible wavelength to the optical detector 290. One or more components of the optical measuring element 205 can be coupled to the processor 226 and/or memory 228. One or more components of the optical measuring element 205 can feed the optical data to the processor 226, and the processor 226 can analyze the optical data to calculate micropost deflection and/or determine one or more clot parameter values.

In these and other embodiments, the measuring element 203 can include a magnetic detection component that is configured to optically measure micropost deflection. For example, FIGS. 8A and 8B are schematic side views of one embodiment of a magnetic measuring element 207 configured in accordance with the present technology. As shown in FIGS. 8A and 8B, each of the microposts 214 can include a magnetic material 270, such as a nanowire, and the magnetic measuring element 207 can include one or more magnetic detectors 272 (e.g., one or more spin valves, Hall probes, fluxgate magnetometers, etc.) that are configured to measure rotation and/or movement of the magnetic material 270 in the deflected microposts 214. FIG. 9, for example, is a graph illustrating spin-valve voltage versus displacement of a deflected micropost 214 containing the magnetic material 270. One or more components of the magnetic measuring element 207 can be coupled to the processor 226 and/or memory 228. One or more components of the magnetic measuring element 207 can feed the magnetic data to the processor 226, and the processor 226 can analyze the magnetic data to calculate micropost deflection and/or determine one or more clot parameter values.

b. Selected Embodiments for Devices, Systems and Methods of Determining Clot Parameters from a Measured Micropost Deflection

It is believed that the aggregated, contracting platelets P exert forces along the vertical length of the micropost 214. As such, deflection measurements can be correlated with a distributed load along a fixed cantilever beam. For example, the clotting force F can be calculated based on micropost deflection δ using the following beam deflection equation:

$$F ( δ ) = \frac{3 π {Ed}^{4}}{64 {h}^{3}} δ \qquad ( 1 )$$

where E is the Young's modulus of the micropost material(s), d is diameter of the micropost 214, and h is the height of the micropost 214. Additionally, the system 200 can include a timer (not shown) that starts when the biological sample is placed in fluid connection with the arrays 221 and stops at a later timepoint whereby at least a portion of the platelets P have adhered to at least one sensing unit 211 in each array 221, aggregated, and caused a deflection of the micropost 214 (e.g., about 40 seconds to about 200 seconds). In some embodiments, the later timepoint can also be great enough to cover the beginning stages of clot lysis. The later timepoint can be predetermined and automatic (e.g., controlled by the processor 226), determined in response to the deflection measurements, and/or manual (e.g., a “stop” button on the analyzer 202). The timer can be coupled to the analyzer 202 and/or processor 226 and the time data can be stored in the memory 228.

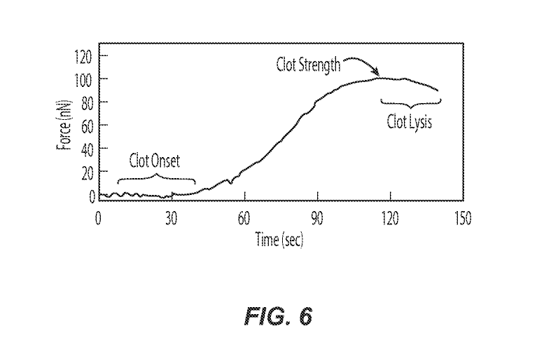

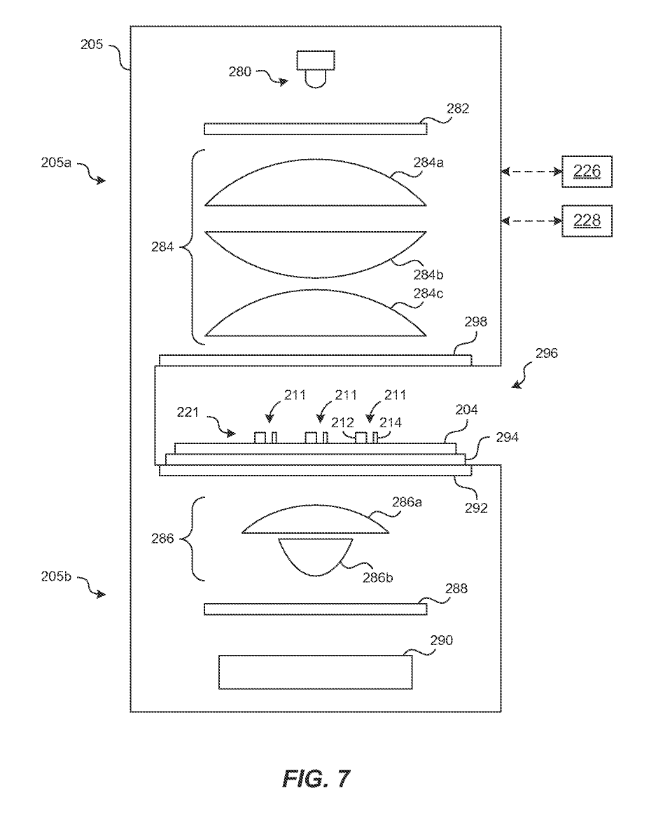

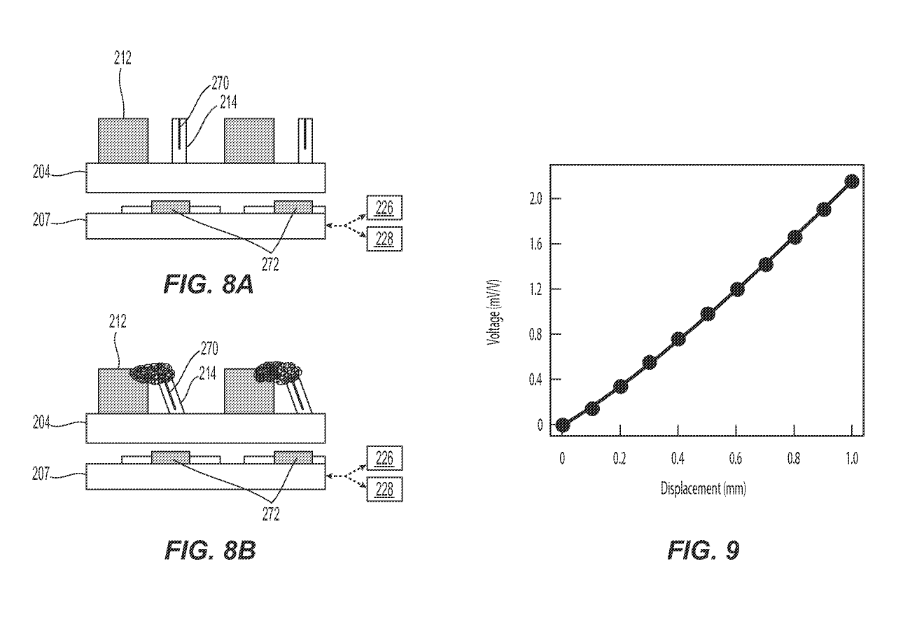

To derive a value for the clot parameters based on the calculated clotting force F (Equation (1)), the processor 226 can correlate the calculated force and recorded time measurements and, based on known relationships between force-time curves and clot parameters, determine a value for one or more of the clot parameters. For example, as shown in the graph of clotting force F versus time in FIG. 6, clot onset is generally the time it takes for the force to show a significant increase, clot strength is generally the maximum recorded force, and clot lysis is generally the time (and/or time period) after the maximum force where there is a significant decrease in force. The processor 226 can indicate one or more of the determined clot parameter values (e.g., via the display 208 (FIG. 2A)). Additionally or alternatively, the processor 226 can generate a force-time curve and display the curve on the display 208.

It can be appreciated that coordination of the delivery of the biological sample to the arrays, the time measurements, and the force measurements can be advantageous to accurate deflection and/or force data. As such, the fluidics device 204 (FIG. 2A) can include a barrier (not shown) that prevents the biological sample from flowing from the inlet 210 (or beginning portion of the inlet channel 216) to the plurality of arrays 221a-e. Accordingly, a clinician can first deliver the biological sample to the inlet 210, and then position the fluidics device 204 in the analyzer 202. The analyzer 202 can include a trigger (e.g., a sharp edge to cut the barrier, a chemical to dissolve the barrier, etc.) that fluidly connects the backed up biological sample with the arrays 221a-e. In other embodiments, the biological sample can be delivered to the fluidics device 204 already positioned at least partially within the analyzer 202. Delivery of the biological sample can trigger the timer to start and/or the clinician can start the timer immediately before delivering the biological sample to the device 204. In yet other embodiments, the timer can be continuously running.

### II. SELECTED EMBODIMENTS OF CLOT ANALYZING SYSTEMS, DEVICES AND METHODS FOR INDIVIDUALIZED MEASUREMENTS, DIAGNOSIS AND/OR TREATMENT

To determine a course of treatment for TIC, currently available coagulation tests (e.g. PT/INR, TEG, etc.) compare one or more of a patient's measured clot parameter value(s) to an average value range based on a large population of patients. For example, if a patient's clot strength is 30, and the group average is 70, then a conventional test would determine that the patient's clot strength is low and the patient should be treated with clot strength agonists, such as adenosine diphosphate (ADP). However, comparing a patient's measured clot parameter value to a group average is not necessarily informative for diagnostic purposes because the values of clot strength, clot onset, and clot lysis can vary greatly from patient to patient. In the example of clot strength given above, if the patient's maximum clot strength is 30, enhancing clot strength with ADP would make no difference, and even worse, fail to address the root cause of TIC (e.g., increased clot lysis and/or delayed clot onset). As such, at least for the purposes of diagnosing TIC, the clot parameter values relative to each individual's maximum and minimum values provide a better assessment of platelet dysfunction than current or measured values alone.

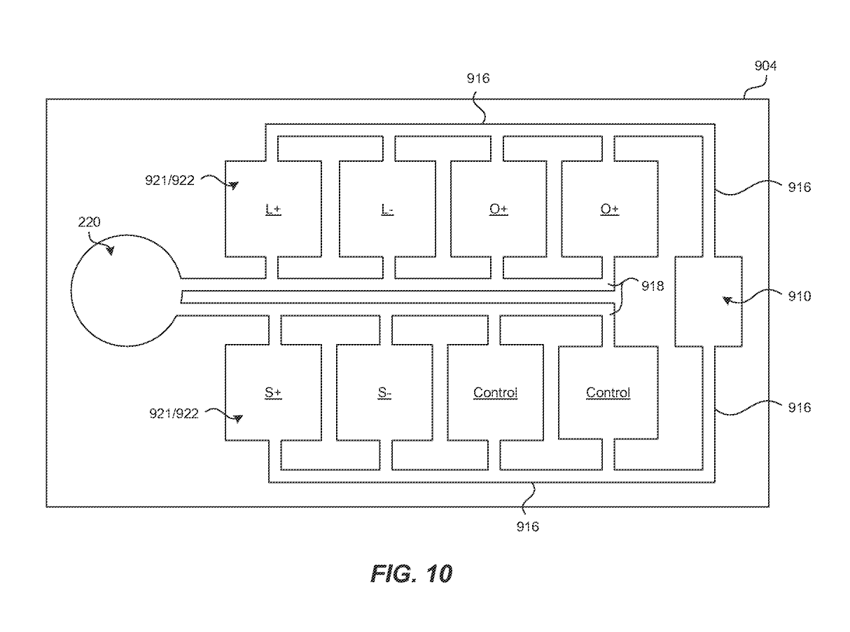

To address these issues, clot analyzing systems configured in accordance with the present technology can include fluidics devices having a plurality of arrays configured to measure a human patient's current value for clot strength, onset, and/or lysis, while also measuring the individual patient's maximum and minimum values of these parameters. For example, FIG. 10 shows a fluidics device 904 for use with the previously described clot analyzing system 200 (FIG. 2A). As shown in FIG. 10, the fluidics device 904 can include eight distinct chambers 922, each housing an array 921 of sensing units 911, and inlet channels 916 for flowing a biological sample into the chambers 922. At least a portion of the sensing units 911, the chambers 922, and/or the inlet channels 916 can be wet or dry-coated with one or more clotting agents configured to effect a biological response in one or more of the clot parameters. For example, the fluidics device 904 can include a control array, an array for measuring a maximum clot lysis value using a clot lysis agonist (L+), an array for measuring a minimum clot lysis value using a clot lysis antagonist (L−), an array for measuring a maximum clot strength value using a clot strength agonist (S+), an array for measuring a minimum clot strength value using a clot strength antagonist (S−), an array for measuring a maximum clot onset value using a clot onset agonist (O+), and/or an array for measuring a minimum clot onset value using a clot onset antagonist (O−).

Although the fluidics device 904 illustrated in FIG. 10 includes eight arrays 921, in other embodiments the device 904 can have more or fewer than eight arrays. For example, the fluidics device 904 can include at least one control array and any one or more of the test or clotting agent arrays (e.g., only the control and the clot lysis antagonist array (and not the agonist array), only the control and the clot onset arrays, all but the clot strength arrays, etc.). Moreover, the fluidics device 904 can also include any number of control arrays (e.g., one, two, three, or more control arrays). For example, the embodiment shown in FIG. 10 utilizes an additional control array to generate a generally constant flow of biological sample to each of the arrays.

The fluidics devices disclosed herein can measure the upper and lower limits of a particular clot parameter using one or more clotting agents. The standardized concentration of each clotting agent can be determined by the following procedure: (1) add the agonist of the particular clotting agent in different concentrations to a set of blood samples (from the same individual) and measure the clot parameter of interest to get the maximum agonist dosage for that clotting agent; (2) add the maximum agonist dosage for the particular clotting agent (calculated in step 1) to different concentrations of antagonists of the particular clotting agent, and measure the clot parameter of interest to get the maximum antagonist dosage for that clotting agent. These measurements can be taken across a large number of patients to determine the standardized concentration for the agonist, and the standardized concentration for the antagonist. The standardized concentration for each agonist and antagonist can then be used for all patients. In other words, even though the clot parameters are measured based on the individual's maximum and minimum clot parameter values (which greatly differ from patient to patient), the clotting agents used in the arrays to get the maximum and minimum clot parameter values are determined based on

Clot strength agonists can include, for example, thrombin, ADP, collagen, vonWillebrand Factor (vWF), fibrinogen, thrombin receptor antagonist (TRAP), epinephrine, ristocetin, and the like. Suitable clot strength antagonists can include, for example, eptifibatide, blebbistatin, platelet inhibitors (aspirin, ADP inhibitors (P2Y12—Clopidogrel, prostaglandins,) thrombin inhibitors (dabigatran), platelet cytoskeletal inhibitors (cytochalasin D, blebbistatin, Platelet 1Balpha inhibitors), and the like. Clot onset agonists include thrombin, tissue factor, collagen, epinephrine, ADP, vWF, coagulation factors (factor VII, prothrombin, Factor X, Factor VIII), Kaolin, and the like. Clot onset antagonists can include, for example, factor Xa inhibitors (rivaroxaban), direct thrombin inhibitors (dabigitran), heparin, low molecular weight heparin, tissue factor pathway inhibitor (TFPI), thrombomodulin, Protein C, Protein S and the like. Clot lysis agonists can include, for example, tissue plasminogen activator (tPA), plasminogen, plasmin, neutrophil elastase, streptokinase, urokinase, and the like. Clot lysis antagonists can include factor XIII, plasminogen activator inhibitor 1 (PAI-1), thrombin-activated fibrinolysis inhibitor (TAFI), antiplasmin, and the like. Additionally, antifibrinolytic drugs can include tranexamic acid, Epsilon aminocaproic acid, aprotinin, and the like.

Referring to FIGS. 10 and 2A together, the fluidics device 904 can be coupled to the analyzer 202, and the measuring element 203 can measure the deflection of the microposts in the arrays 921 and transfer this information to the processor 226 (as previously described). The processor 226 can then determine the clot parameter values for each array 921 (as previously described) and systematically compare the control values to the maximum and minimum values for each measured clot parameter. This way, the processor can formulate an individualized clot parameter measurement for each patient based on that patient's maximum and minimum clot parameter values.

Based on the comparison between the current values and the maximum and/or minimum values of the clot parameter(s), the display 208 (FIG. 2A) can indicate to the clinician the current, measured value for one or more clot parameters, as well as the maximum and/or minimum values of one or more clot parameters. For example, the display 208 can indicate a patient's current clot strength value, current clot lysis value, current clot onset value, maximum clot strength value, maximum clot lysis value, maximum clot onset value, minimum clot strength value, minimum clot lysis value, minimum clot onset value, and/or any derivatives of any of the preceding parameters.

The display 208 (via instructions from the processor 206) can also indicate a TIC diagnosis and/or suggested course of treatment based on the comparison between the current values and the maximum and/or minimum values for each measured clot parameter. Likewise, in some embodiments the display 208 can indicate the clot parameter values to inform the clinician's decision on course of treatment. For example, if the detected clot onset time and strength values are normal and the clot lysis value has increased, the clinician can specifically treat the patient with an antifibrinolytic agent. An antifibrinolytic agent interferes with the formation of the fibrinolytic enzyme plasmin so that there is less plasmin to destroy the fibrin mesh surrounding the platelet plug (see FIG. 1), thus slowing or weakening the clot lysis process. As another example, if the clot onset value is normal, but the clot strength value is low and the clot lysis value has increased, then the clinician can specifically treat the patient with a platelet transfusion and plasma (to increase clot strength) and an antifibrinolytic agent (to reduce clot lysis). If all parameter values are abnormal (i.e., prolonged clot onset, low clot strength, and increased clot lysis), the clinician can treat with coagulation factors (prothrombin complex concentrate or plasma), fibrinogen and/or platelet transfusion, and an antifibrinolytic agent. If any one of the above are present in isolation, and there is ongoing bleeding, the clinician can use the specific therapy to restore clot onset, strength, or lysis.

Conventional devices can take 30 minutes to an hour and a half to determine a clot parameter value, and even then the value is not necessarily helpful in identifying a meaningful course of treatment. The clot analyzing system 200 of the present technology can determine individualized clot parameter values, and specify a course of treatment, in three minutes or less.

### III. MATERIALS AND METHODS FOR MICROSTRUCTURE FABRICATION

The microstructures of the sensing units (e.g., the microblocks 212 and microposts 214 illustrated in FIG. 2C) can be fabricated using a negative mold. The negative mold can be fabricated using established contact photolithography on a silicon wafer using separate layers of SU-8 (Microchem) series photoresist. Chrome masks can be used to build each layer which results in a permanent positive SU-8 master structure. The surface can be silanized (tridecafluoro-1,1,2,2-tetrahydrooctyl)-1-trichlorosilane (T2492-KG, United Chemical Technologies), for example, to prevent adhesion of the microstructure material.

The flexible and rigid microstructures of the present technology can be made of PDMS and built using soft lithography in a two-step replicate fabrication process. For example, PDMS can be mixed with its curing agent at a 10:1 ratio, degassed, and poured onto the positive SU-8 master structure. The structure can then be cured in an oven at 110° C. for 20 minutes to produce a negative mold from the master structure. The negative mold can then be plasma treated (e.g., Plasma Prep II, SPI) for about 90 seconds to activate the surface, then silane treated under vacuum to passivate the surface. A 10:1 PDMS can then be applied to the negative, before setting the negative against a cleaned coverglass (e.g., no. 0) and cured in an oven at 110° C. for 24 hours. The negative can later be removed, thus leaving a PDMS microstructure device that is a replicate of the original SU-8 master structure. A continuous PDMS manifold having inlet and outlet ports in a flat PDMS block can be plasma treated and pressed into place on the microchannel. This creates an irreversible, watertight bond between the two surfaces, and forms a rectangular duct path with ports at either end and the sensors in the middle.

It will be appreciated that the above materials and methods are provided by way of example and should not be construed to limit the materials and/or manufacturing methods of the present technology.

### IV. EXAMPLES

The following examples are illustrative of several embodiments of the present technology: 1. A system for analyzing a biological sample, comprising:
a plurality of arrays of microstructures, wherein each microstructure includes a generally rigid structure and a generally flexible structure, and wherein the plurality of arrays includes—
   a test array configured to be in fluid connection with a clotting agent, wherein the clotting agent is configured to effect a biological response in a clot parameter of the biological sample;
   a control array that is not in fluid connection with the clotting agent;
a plurality of fluid channels configured to receive the biological sample, wherein at least a portion of the fluid channels are sized to house one of the arrays; and
a measuring element configured to detect a degree of deflection of one or more of the flexible structures in one or more of the arrays.

2. The system of example 1 wherein the clot parameter is selected from clot strength, clot lysis, and clot onset.

3. The system of any of examples 1 or 2 wherein the clotting agent is an agonist or an antagonist of the clot parameter.

4. The system of any of examples 1-3 wherein the microstructures of the test array are at least partially coated with the first clotting agent.

5. The system of any of examples 1-4 wherein the plurality of fluid channels include—
an inlet channel;
a chamber fluidly coupled to the inlet channel, wherein the test array is in the chamber;
wherein—
   at least one of the microstructures of the test array, the inlet channel, and/or the chamber are at least partially coated with the clotting agent.

6. The system of any of examples 1-5 wherein the generally rigid structure has a rectangular shape, and the generally flexible structure has a cylindrical shape.

7. The system of any of examples 1-6 wherein the measuring element comprises an optical detection component and/or a magnetic detection component.

8. A system for analyzing a biological sample, comprising:
a plurality of arrays of microstructures, wherein each microstructure includes a generally rigid structure and a generally flexible structure, and wherein the plurality of arrays includes—
   a first array configured to be in fluid connection with a first clotting agent, wherein the first clotting agent is configured to effect a biological response in a clot parameter of the biological sample;
   a second array configured to be in fluid connection with a second clotting agent, wherein the second clotting agent is configured to effect a biological response in the clot parameter, and wherein the second clotting agent is different than the first clotting agent; and
   a third array that is not in fluid connection with the first clotting agent or the second clotting agent;
a plurality of fluid channels configured to receive the biological sample, wherein at least a portion of the fluid channels are sized to house one of the arrays; and
a measuring element configured to detect a degree of deflection of one or more of the flexible structures in one or more of the arrays.

9. The system of example 8 wherein the clot parameter is selected from clot strength, clot lysis, and clot onset.

10. The system of any of examples 8 or 9 wherein the first clotting agent is an agonist of the clot parameter and the second clotting agent is an antagonist of the clot parameter.

11. The system of any of examples 8-10 wherein:
the microstructures of the first array are at least partially coated with the first clotting agent, and wherein the first clotting agent is an antagonist; and
the microstructures of the second array are at least partially coated with the second clotting agent, and wherein the second clotting agent is an agonist.

12. The system of any of examples 8-10 wherein the plurality of fluid channels include—
a first inlet channel;
a first chamber fluidly coupled to the first inlet channel, wherein the first array is in the first chamber;
a second inlet channel;
a second chamber fluidly coupled to the second inlet channel, wherein the second array is in the second chamber; and
wherein—
   at least one of the microstructures of the first array, the first inlet channel, and/or the first chamber are at least partially coated with the first clotting agent; and
   at least one of the microstructures of the second array, the second inlet channel, and/or the second inlet chamber are at least partially coated with the second clotting agent.

13. The system of any of examples 8-12 wherein the generally rigid structure has a rectangular shape, and the generally flexible structure has a cylindrical shape.

14. The system of any of examples 8-13 wherein the measuring element comprises an optical detection component and/or a magnetic detection component.

15. The system of any of examples 8-14 wherein the measuring element comprises a magnetic detection component is a spin valve, a Hall probe, and/or a fluxgate magnetometer.

16. The system of example 15 wherein individual generally flexible structures include a magnetic material.

17. The system of any of examples 15 or 16 wherein the magnetic detection component comprises spin valves positioned between the individual generally rigid structures and generally flexible structures, and wherein the spin valves are configured to detect changes in a magnetic field in the array caused by deflection of the generally flexible structures including the magnetic material.

18. The system of any of examples 8-14 wherein the measuring element comprises an optical detection component that is one of a phase contrast microscope, a fluorescence microscope, a confocal microscope, or a photodiode.

19. The system of any of examples 8-18 wherein the biological sample comprises whole blood, platelets, endothelial cells, circulating tumor cells, cancer cells, fibroblasts, smooth muscle cells, cardiomyocytes, red blood cells, white blood cells, bacteria, megakaryocytes, and/or fragments thereof.

20. The system of any of examples 8-19 wherein at least some of the microstructures are at least partially coated with at least one binding element selected from a group consisting of proteins, glycans, polyglycans, glycoproteins, collagen, von Willebrand factor, vitronectin, laminin, monoclonal antibodies, polyclonal antibodies, plasmin, agonists, matrix proteins, inhibitors of actin-myosin activity, and fragments thereof.

21. The system of any of examples 8-20, further comprising a display configured to display a characteristic of the biological sample based on the degree of deflection of the one or more generally flexible structures.

22. The system of any of examples 8-21, wherein:
the clot parameter is clot strength;
the first clotting agent is adenosine diphosphate (ADP); and
the second clotting agent is selected from eptifibatide and blebbistatin.

23. The system of any of examples 8-22, wherein:
the clot parameter is clot onset;
the first clotting agent is bivalrudin; and
the second clotting agent is at least one of thrombin or tranexamix acid.

24. The system of any of examples 8-23, wherein:
the clot parameter is clot lysis; and
the first clotting agent is tissue plasminogen activator (tPA).

25. The system of any of examples 8-24 wherein the clot parameter is a first clot parameter, and wherein the system further includes:
a fourth array configured to be in fluid connection with a third clotting agent, wherein the third clotting agent is configured to effect a biological response in a second clot parameter of the biological sample; and
a fifth array configured to be in fluid connection with a fourth clotting agent, wherein the fourth clotting agent is configured to effect a biological response in the second clot parameter, and wherein the fourth clotting agent is different than the third clotting agent.

26. The system of example 25, further including:
a sixth array configured to be in fluid connection with a fifth clotting agent, wherein the fifth clotting agent is configured to effect a biological response in a third clot parameter of the biological sample; and
a seventh array configured to be in fluid connection with a sixth clotting agent, wherein the sixth clotting agent is configured to effect a biological response in the third clot parameter, and wherein the sixth clotting agent is different than the fifth clotting agent.

27. A method, comprising:
receiving a biological sample of a human patient through a network of microchannels;
flowing at least a portion of the biological sample over a first array of sensing units and a second array of sensing units, wherein—
   each sensing unit of the first array includes a first generally rigid microstructure and a first generally flexible microstructure, and
   each sensing unit of the second array includes a second generally rigid microstructure and a second generally flexible microstructure;
detecting movement of the first generally flexible microstructure relative to the corresponding first generally rigid microstructure in response to the biological sample;
detecting movement of the second generally flexible microstructure relative to the corresponding second generally rigid microstructure in response to the biological sample;
determining a current value of a clot parameter of the biological sample based on the detected movement of the first generally flexible microstructure; and
determining at least one of a maximum value and a minimum value of the clot parameter based on the detected movement of the second generally flexible microstructure.

28. The method of example 27, further comprising comparing the current value to at least one of the maximum value and the minimum value.

29. The method of example 28, further comprising identifying a course of treatment based on the comparison.

30. The method of example 27, further comprising introducing a clotting agent to the second array.

31. The method of example 27, further comprising indicating at least one of the current value, the maximum value, and/or the minimum value of the clot parameter.

32. The method of example 27 wherein the clot parameter is selected from clot lysis, clot onset, and clot strength.

33. A method, comprising:
receiving a biological sample of a human patient through a network of microchannels;
flowing at least a portion of the biological sample over a first, second and third array of sensing units, wherein—
   each sensing unit of the first array includes a first generally rigid microstructure and a first generally flexible microstructure;
   each sensing unit of the second array includes a second generally rigid microstructure and a second generally flexible microstructure;
   each sensing unit of the third array includes a third generally rigid microstructure and a third generally flexible microstructure;
detecting—
   movement of the first generally flexible microstructure relative to the corresponding first generally rigid microstructure in response to the biological sample;
   movement of the second generally flexible microstructure relative to the corresponding second generally rigid microstructure in response to the biological sample; and
   movement of the third generally flexible microstructure relative to the corresponding third generally rigid microstructure in response to the biological sample;
determining—
   a current value of a clot parameter of the biological sample based on the detected movement of the first generally flexible microstructure;
   a minimum value of the clot parameter based on the detected movement of the second generally flexible microstructure; and
   a maximum value of the clot parameter based on the detected movement of the third generally flexible microstructure.

34. The method of example 34, further comprising comparing the current value to the maximum value and the minimum value.

### V. CONCLUSION

As used herein and unless otherwise indicated, the terms “a” and “an” are taken to mean “one,” “at least one” or “one or more.” Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to.” Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

All of the references cited herein are incorporated by reference in their entireties. Such references include the following pending applications: (a) U.S. Provisional Patent Application No. 61/645,191, filed May 10, 2012; (b) U.S. Provisional Patent Application No. 61/709,809, filed Oct. 4, 2012; (c) U.S. patent application Ser. No. 13/663,339, filed Oct. 29, 2012; (d) PCT Application No. PCT/US2013/031782 filed Mar. 14, 2013; and (e) U.S. Provisional Patent Application No. 61/760,849, filed Feb. 5, 2013.

Aspects of the disclosure can be modified, if necessary, to employ the systems, functions, and concepts of the above references and application to provide yet further embodiments of the disclosure. These and other changes can be made to the disclosure in light of the detailed description.

The technology disclosed herein offers several advantages over existing systems. For example, the devices disclosed herein can quickly and accurately detect platelet function in emergency point of care settings. The devices can be portable, battery operated, and require little to no warm-up time. A sample need only be a few microliters and can be tested in less than five minutes. Further, the device can be relatively simple, with no moving parts that could mechanically malfunction and no vibration or centrifuge required. Further, such a simple device can be manufactured relatively inexpensively.

From the foregoing it will be appreciated that, although specific embodiments of the technology have been described herein for purposes of illustration, various modifications may be made without deviating from the spirit and scope of the technology. Further, certain aspects of the new technology described in the context of particular embodiments may be combined or eliminated in other embodiments. Moreover, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein. Thus, the disclosure is not limited except as by the appended claims.

I/We claim:

1. A system for analyzing a biological sample, comprising:
a plurality of arrays of microstructures, wherein each microstructure includes a generally rigid structure and a generally flexible structure, and wherein the plurality of arrays includes—
a test array configured to be in fluid connection with a clotting agent, wherein the clotting agent is configured to effect a biological response in a clot parameter of the biological sample;
a control array that is not in fluid connection with the clotting agent;
a plurality of fluid channels configured to receive the biological sample, wherein at least a portion of the fluid channels are sized to house one of the arrays; and
a measuring element configured to detect a degree of deflection of one or more of the flexible structures in one or more of the arrays.

2. The system of claim 1 wherein the clot parameter is selected from clot strength, clot lysis, and clot onset.

3. The system of claim 1 wherein the clotting agent is an agonist or an antagonist of the clot parameter.

4. The system of claim 1 wherein the microstructures of the test array are at least partially coated with the first clotting agent.

5. The system of claim 1 wherein the plurality of fluid channels include—
an inlet channel;
a chamber fluidly coupled to the inlet channel, wherein the test array is in the chamber;
wherein—
at least one of the microstructures of the test array, the inlet channel, and/or the chamber are at least partially coated with the clotting agent.

6. The system of claim 1 wherein the generally rigid structure has a rectangular shape, and the generally flexible structure has a cylindrical shape.

7. The system of claim 1 wherein the measuring element comprises an optical detection component and/or a magnetic detection component.

8. A system for analyzing a biological sample, comprising:
a plurality of arrays of microstructures, wherein each microstructure includes a generally rigid structure and a generally flexible structure, and wherein the plurality of arrays includes—
a first array configured to be in fluid connection with a first clotting agent, wherein the first clotting agent is configured to effect a biological response in a clot parameter of the biological sample;
a second array configured to be in fluid connection with a second clotting agent, wherein the second clotting agent is configured to effect a biological response in the clot parameter, and wherein the second clotting agent is different than the first clotting agent; and
a third array that is not in fluid connection with the first clotting agent or the second clotting agent;
a plurality of fluid channels configured to receive the biological sample, wherein at least a portion of the fluid channels are sized to house one of the arrays; and
a measuring element configured to detect a degree of deflection of one or more of the flexible structures in one or more of the arrays.

9. The system of claim 8 wherein the clot parameter is selected from clot strength, clot lysis, and clot onset.

10. The system of claim 8 wherein the first clotting agent is an agonist of the clot parameter and the second clotting agent is an antagonist of the clot parameter.

11. The system of claim 8 wherein:
the microstructures of the first array are at least partially coated with the first clotting agent, and wherein the first clotting agent is an antagonist; and
the microstructures of the second array are at least partially coated with the second clotting agent, and wherein the second clotting agent is an agonist.

12. The system of claim 8 wherein the plurality of fluid channels include—
a first inlet channel;
a first chamber fluidly coupled to the first inlet channel, wherein the first array is in the first chamber;
a second inlet channel;
a second chamber fluidly coupled to the second inlet channel, wherein the second array is in the second chamber; and
wherein—
at least one of the microstructures of the first array, the first inlet channel, and/or the first chamber are at least partially coated with the first clotting agent; and
at least one of the microstructures of the second array, the second inlet channel, and/or the second inlet chamber are at least partially coated with the second clotting agent.

13. The system of claim 8 wherein the generally rigid structure has a rectangular shape, and the generally flexible structure has a cylindrical shape.

14. The system of claim 8 wherein the measuring element comprises an optical detection component and/or a magnetic detection component.

15. The system of claim 8 wherein the measuring element comprises a magnetic detection component is a spin valve, a Hall probe, and/or a fluxgate magnetometer.

16. The system of claim 15 wherein individual generally flexible structures include a magnetic material.

17. The system of claim 15 wherein the magnetic detection component comprises spin valves positioned between the individual generally rigid structures and generally flexible structures, and wherein the spin valves are configured to detect changes in a magnetic field in the array caused by deflection of the generally flexible structures including the magnetic material.

18. The system of claim 8 wherein the measuring element comprises an optical detection component that is one of a phase contrast microscope, a fluorescence microscope, a confocal microscope, or a photodiode.

19. The system of claim 8 wherein the biological sample comprises whole blood, platelets, endothelial cells, circulating tumor cells, cancer cells, fibroblasts, smooth muscle cells, cardiomyocytes, red blood cells, white blood cells, bacteria, megakaryocytes, and/or fragments thereof.

20. The system of claim 8 wherein at least some of the microstructures are at least partially coated with at least one binding element selected from a group consisting of proteins, glycans, polyglycans, glycoproteins, collagen, von Willebrand factor, vitronectin, laminin, monoclonal antibodies, polyclonal antibodies, plasmin, agonists, matrix proteins, inhibitors of actin-myosin activity, and fragments thereof.

21. The system of claim 8, further comprising a display configured to display a characteristic of the biological sample based on the degree of deflection of the one or more generally flexible structures.

22. The system of claim 8, wherein:
the clot parameter is clot strength;
the first clotting agent is adenosine diphosphate (ADP); and
the second clotting agent is selected from eptifibatide and blebbistatin.

23. The system of claim 8, wherein:
the clot parameter is clot onset;
the first clotting agent is bivalrudin; and
the second clotting agent is at least one of thrombin or tranexamix acid.

24. The system of claim 8, wherein:
the clot parameter is clot lysis; and
the first clotting agent is tissue plasminogen activator (tPA).

25. The system of claim 8 wherein the clot parameter is a first clot parameter, and wherein the system further includes:
a fourth array configured to be in fluid connection with a third clotting agent, wherein the third clotting agent is configured to effect a biological response in a second clot parameter of the biological sample; and
a fifth array configured to be in fluid connection with a fourth clotting agent, wherein the fourth clotting agent is configured to effect a biological response in the second clot parameter, and wherein the fourth clotting agent is different than the third clotting agent.

26. The system of claim 8, further including:
a sixth array configured to be in fluid connection with a fifth clotting agent, wherein the fifth clotting agent is configured to effect a biological response in a third clot parameter of the biological sample; and
a seventh array configured to be in fluid connection with a sixth clotting agent, wherein the sixth clotting agent is configured to effect a biological response in the third clot parameter, and wherein the sixth clotting agent is different than the fifth clotting agent.

27. A method, comprising:
receiving a biological sample of a human patient through a network of microchannels;
flowing at least a portion of the biological sample over a first array of sensing units and a second array of sensing units, wherein—
each sensing unit of the first array includes a first generally rigid microstructure and a first generally flexible microstructure, and
each sensing unit of the second array includes a second generally rigid microstructure and a second generally flexible microstructure;
detecting movement of the first generally flexible microstructure relative to the corresponding first generally rigid microstructure in response to the biological sample;
detecting movement of the second generally flexible microstructure relative to the corresponding second generally rigid microstructure in response to the biological sample;
determining a current value of a clot parameter of the biological sample based on the detected movement of the first generally flexible microstructure; and
determining at least one of a maximum value and a minimum value of the clot parameter based on the detected movement of the second generally flexible microstructure.

28. The method of claim 27, further comprising comparing the current value to at least one of the maximum value and the minimum value.

29. The method of claim 28, further comprising identifying a course of treatment based on the comparison.

30. The method of claim 27, further comprising introducing a clotting agent to the second array.

31. The method of claim 27, further comprising indicating at least one of the current value, the maximum value, and/or the minimum value of the clot parameter.

32. The method of claim 27 wherein the clot parameter is selected from clot lysis, clot onset, and clot strength.

33. A method, comprising:
receiving a biological sample of a human patient through a network of microchannels;
flowing at least a portion of the biological sample over a first, second and third array of sensing units, wherein—
each sensing unit of the first array includes a first generally rigid microstructure and a first generally flexible microstructure;
each sensing unit of the second array includes a second generally rigid microstructure and a second generally flexible microstructure;
each sensing unit of the third array includes a third generally rigid microstructure and a third generally flexible microstructure;
detecting—
movement of the first generally flexible microstructure relative to the corresponding first generally rigid microstructure in response to the biological sample;
movement of the second generally flexible microstructure relative to the corresponding second generally rigid microstructure in response to the biological sample; and
movement of the third generally flexible microstructure relative to the corresponding third generally rigid microstructure in response to the biological sample;
determining—
a current value of a clot parameter of the biological sample based on the detected movement of the first generally flexible microstructure;
a minimum value of the clot parameter based on the detected movement of the second generally flexible microstructure; and
a maximum value of the clot parameter based on the detected movement of the third generally flexible microstructure.

34. The method of claim 34, further comprising comparing the current value to the maximum value and the minimum value.
